(19) United States
(12) Patent Application Publication
(10) Pub. No.: US 2015/0087955 A1
(43) Pub. Date: Mar. 26, 2015

(54) MULTI-WAVELENGTH PHOTOACOUSTIC CHARACTERIZATION AND THERMOGRAPHIC IMAGING

(71) Applicant: BOARD OF REGENTS, THE UNIVERSITY OF TEXAS SYSTEM, Austin, TX (US)

(72) Inventors: Stanislav Emelianov, Austin, TX (US); Nicholas Dana, Austin, TX (US); Richard Bouchard, Houston, TX (US); Luigi Di Biase, Austin, TX (US); Andrea Natale, Austin, TX (US)

(21) Appl. No.: 14/495,073

(22) Filed: Sep. 24, 2014

Related U.S. Application Data
(60) Provisional application No. 61/881,743, filed on Sep. 24, 2013.

(51) Int. Cl. A61B 5/00; A61B 18/08; A61B 5/02

(52) U.S. Cl. CPC A61B 5/0095; A61B 5/4244; A61B 5/4381; A61B 5/02028; A61B 5/743; A61B 18/08; A61B 2018/00577
USPC 600/407; 606/27; 435/29

(57) ABSTRACT

The present disclosure relates to a method comprising imaging a tissue with multi-wavelength photoacoustic imaging, to provide a visualization, characterization and/or thermographic imaging of the tissue.

### CROSS-REFERENCE TO RELATED APPLICATIONS

This Application claims the benefit of U.S. Provisional Application Ser. No. 61/881,743 filed on Sep. 24, 2013, the entirety of which is incorporated by reference.

### STATEMENT OF GOVERNMENT INTEREST

This invention was made with support under Award No. EB007507-03, awarded by National Institute of Health. The U.S. government has certain rights in the invention.

### BACKGROUND

Atrial fibrillation (AF) is currently the most common arrhythmia encountered in clinical practice, with estimates as high as 6.1 million sufferers in 2010 in the United States alone. AF has been implicated in an increased risk of stroke, dementia, heart failure and an excess mortality rate. Anti-arrhythmic drugs are burdened with significant side effects, toxicity and poor capability to maintain normal sinus rhythm and thus are not the ideal treatment option for AF patients. Radio-frequency (RF) trans-catheter ablation is currently the most effective treatment for AF as it can isolate the firing of ectopic foci, typically located around the pulmonary veins. Unfortunately, RF ablation lesions can vary considerably with catheter contact force, orientation, size and RF energy parameters. Lesion “reconnection” and “recovery” has been a major cause for procedural failure and can necessitate repeat procedures. Furthermore, ablation procedures carry the risk of severe complications, such as atrio-esophageal fistulae or cardiac tamponade. These limitations of RF trans-catheter ablation will not be significantly improved without a real-time (RT) tool to characterize lesions intraoperatively.

### DRAWINGS

The patent or application file contains at least one drawing executed in color. Copies of this patent or patent application publication with color drawing(s) will be provided by the Office upon request and payment of the necessary fee.

A more complete understanding of this disclosure may be acquired by referring to the following description taken in combination with the accompanying figures in which:

FIG. 1 shows a schematic of combined PA/US imaging system.

FIG. 2 shows single-wavelength PA image (710 nm) overlaying US image of a cardiac tissue sample with Ablated and Non-ablated ROIs (A); mean ROI photoacoustic signal plotted vs. wavelength (B); tissue characterization map (TCM) (C); and reference spectra for Ablated (averaged over eight samples from two hearts) and Non-ablated (derived from optical extinction data from deoxy-Hb) tissue (D).

FIG. 3 shows top-view (A) and side-view (C) stained sample gross pathology with sample-boundary (blue) and ablated-region (red) segmentation; and matching top—(B) and side-view (D) TCM-US images with segmented ablated region (red).

FIG. 4 shows three-dimensional rendering (A) of TCM volume with clipping plane corresponding to tissue bisection (B); and matching top—(C) and side-view (D) gross pathology photographs with axes and FOVs indicated by arrows and boxes, respectively.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as defined by the appended claims.

### DESCRIPTION

The present disclosure generally relates to methods comprising imaging a tissue with multi-wavelength photoacoustic imaging, to provide a three-dimensional visualization, characterization and/or thermographic imaging of the tissue, and systems thereof.

Photoacoustic (PA) imaging is an imaging technique utilizing short-duration laser pulses which are absorbed by chromophores (such as deoxy-hemoglobin [Hb]) in the tissue, resulting in thermoelastic expansion and generation of an acoustic transient. These local transients can be imaged using a traditional US transducer, providing an optical absorption map with resolutions on the order of tens of micrometers (or hundreds of micrometers using a 7.5 MHz US probe, which is common for EP intracardiac or transesophageal US applications) and at imaging depths in excess of a centimeter.

The peak photoacoustic pressure, generated during thermal and stress confinement, is commonly modeled as

$${p}_{0} ( r , λ ) = ( \frac{β ( T ) {v}_{s}^{2} ( T )}{{C}_{p} ( T )} ) {μ}_{abs} ( r , λ ) Φ ( r , λ ) \qquad Eq . 1$$

where β(T) [K⁻¹] is the temperature dependent thermal coefficient of volume expansion, νₛ(T) [cm s⁻¹] is the sound velocity in tissue, Cₚ(T) [J kg⁻¹ K⁻¹] is the heat capacity at constant pressure, μ_{abs}(r, λ) [cm⁻¹] is the optical absorption coefficient and Φ(r, λ) [J cm⁻²] is the local optical fluence. As such, r, λ and T represent spatial distribution, optical wavelength and temperature, respectively.

PA imaging contrast is provided by variations in optical absorption (μ_{abs}) resulting from variations in the concentration of endogenous or exogenous chromophores. Due to the wavelength dependence of μ_{abs}, multi-wavelength photoacoustic imaging can be performed for tissue characterization purposes. PA imaging's reliance upon ultrasonic sensing allows straightforward co-registration with anatomical US images, providing molecularly-sensitive anatomical PA/US images. For these reasons, PA imaging is a powerful medical imaging modality in cancer detection, disease staging, and therapy guidance.

The present disclosure provides, according to certain embodiments, methods comprising imaging a tissue with multi-wavelength photoacoustic imaging, to provide a three-dimensional visualization, characterization, and/or thermographic imaging of the tissue. The present disclosure also provides systems and components for imaging a tissue with multi-wavelength photoacoustic imaging. In certain embodiments, the multi-wavelength photoacoustic imaging may be spectroscopic photoacoustic imaging [sPA].

In certain embodiments, the tissue imaged is a cardiac tissue, a liver tissue, or a prostate tissues.

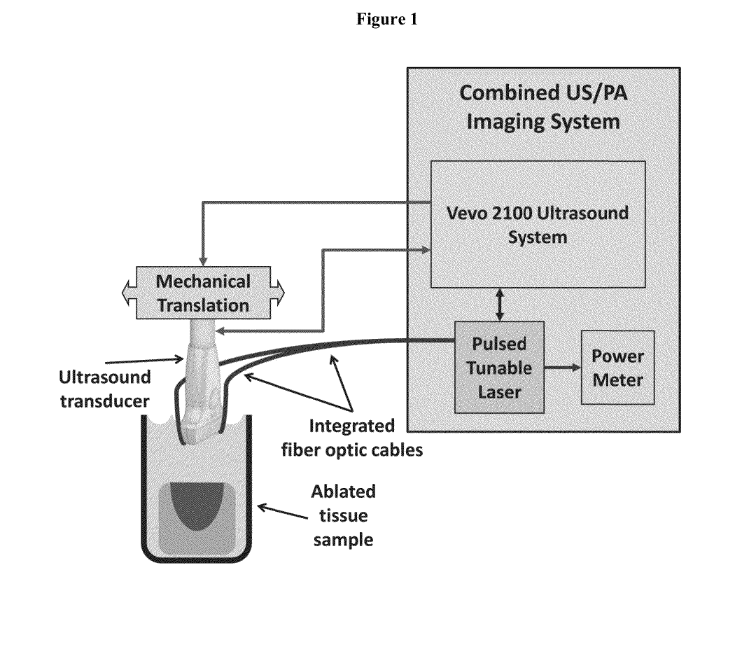

In another embodiment, the method further comprises applying ultrasound imaging to the tissue. See, e.g., FIG. 1.

In another embodiment, the method further comprises imaging the tissue with a single-wavelength photoacoustic imaging. In a further embodiment, the single-wavelength photoacoustic imaging occurs before the multi-wavelength photoacoustic imaging.

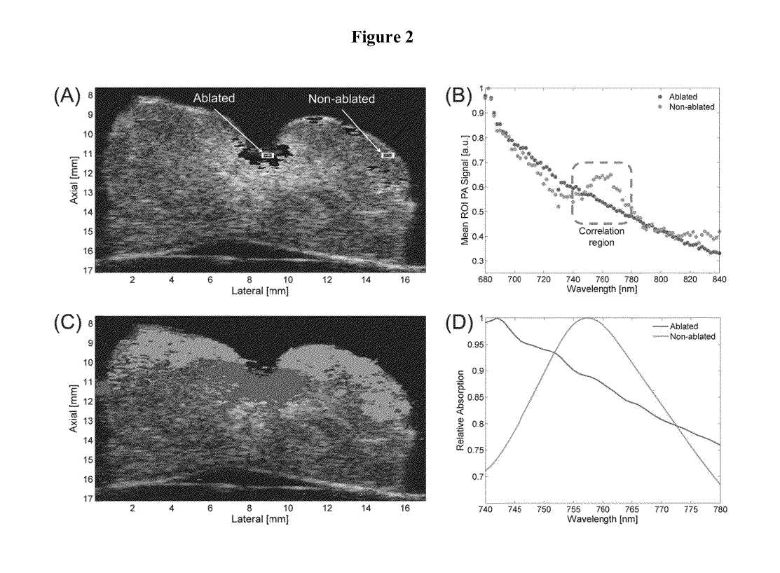

Single-wavelength PA imaging shows high contrast from the lesion core (see, e.g., FIG. 2A), which indicates it may be useful as a pilot scan to locate the approximate lesion center. The sPA based tissue characterization map (TCM) demonstrated the ability to reliably identify ablated and non-ablated myocardium with high accuracy and sub-millimeter precision (FIGS. 2C, 3B, 3D, 4A and 4B). TCM images of ablated and non-ablated tissue appear relatively uniform across the entire imaged area or volume (FIGS. 2C, 3B, 3D, 4A and 4B). All TCM images showed consistent tissue characterization and correlated well to matched images of gross pathology to at least 2 mm in depth (FIGS. 2C, 3 and 4).

In another embodiment, the method further comprising processing the multi-wavelength photoacoustic imaging data to form 3-D sPA data. In a further embodiment, the method further comprising generating a TCM from the 3-D sPA data.

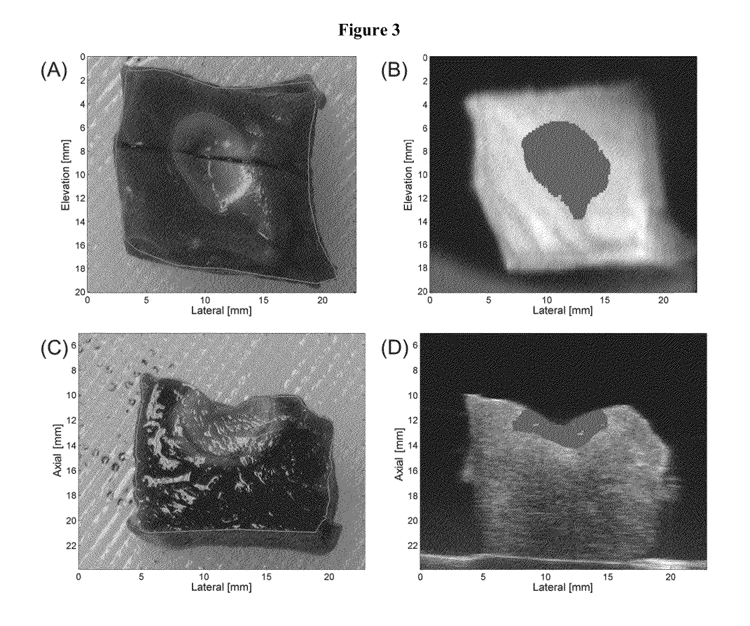

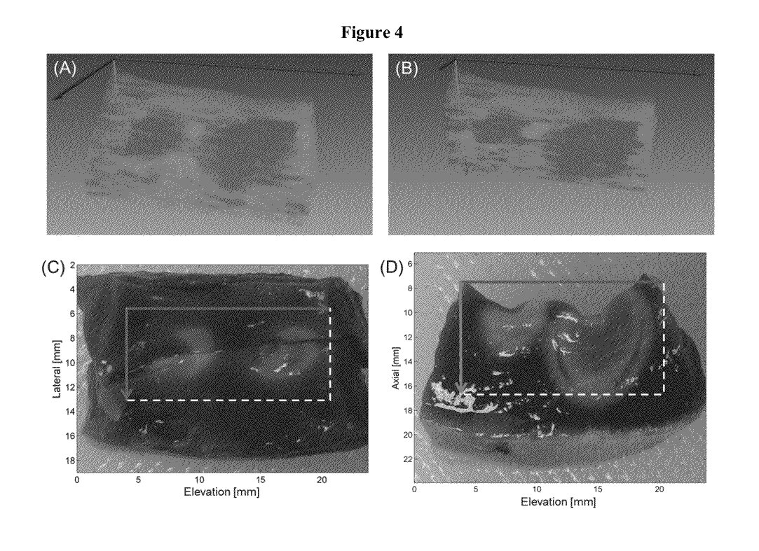

One caveat of PA imaging is that imaging artifacts, resulting from refractive index discontinuities, can occasionally be seen near tissue boundary locations. These artifacts, however, would be minimal in an in vivo environment, as the imaging target would not be an explanted tissue sample with tissue-solution boundaries. Also, spectroscopic-based images, such as a TCM, may further reduce artifacts by correlating multiple wavelengths and reducing the effects of saturated voxel regions by identifying absorbers. Given the 3-D datasets (FIGS. 3B, 3D, 4A and 4B) were acquired using only six averages and without laser energy pulse-to-pulse normalization, increased frame averaging, improved energy normalization and increased surface fluence should improve TCM quality and increase depth penetration beyond 3 mm, for many applications beyond atrial ablation. When considering the segmentation metrics, it should be first noted that a basic rigid registration method was utilized that did not account for tissue deformation, which will inherently introduce error. See Table 1. Despite this limitation, excellent agreement was achieved with the top-view comparisons, with no elevation and small lateral offset observed. Furthermore, the average top-view segmented areas differed only by 4 mm², yielding a % Area Agreement of 69±11%; this area agreement is comparable with other lesion imaging methods currently being developed.

For the side-view orientation, lateral offset was improved when compared to the top-view orientation (0.1 mm vs. to 0.4 mm). The relatively high axial offset (1.2 mm) observed in this orientation is attributable to the limited ability in this initial implementation of sPA imaging to characterize tissue at depths greater than 3 mm; this penetration depth can be increased with improved frame averaging, improved energy normalization and increased laser fluence. With said improvements the ability of this imaging modality to reliably characterize tissue and assess transmurality at depths >3 mm could be determined. As can be seen in

FIGS. 3C and 4D, the ablated regions extend axially from approximately 10 mm to 15 mm and 11 mm to 16 mm, respectively (5 mm in both instances). The deepest 2 mm of ablated myocardium was not reliably visualized, so the axial position of the segmented TCM centroid naturally appears shallow compared to the centroid of the segmented lesion from photographed gross pathology. This also accounts for the lower % Area Agreement score (36%) when compared with the top-view results (69%).

The 3-D rendering (FIGS. 4A and 4B) of the dual-lesion data gave the opportunity to examine the ability of the system to identify adjacent lesions. We can clearly see two distinct regions, approximately 4 mm in diameter each, which correlate strongly to the Ablated reference (red); surrounding these two regions is tissue that consistently correlates to the Non-ablated reference (blue). This segmentation agrees well with gross pathology images, shown in FIGS. 4C and 4D. Additionally, a small region of tissue that correlates to both reference spectra is seen lying between the two lesions in FIG. 4B (green). This segmentation agrees well with matching gross pathology (FIG. 4D), as a small isthmus of ambiguous tissue lies between the two lesions (approximately 11 mm in the axial and elevation dimensions). This result emphasizes the ability of sPA to identify regions where lesion contiguity may be in question.

Given that the PA spectrum observed in ablated tissue results from bulk hyperthermia-induced protein denaturation, resulting from tissue heating, it is believed that this spectrum would not vary significantly from patient to patient, nor vary significantly between normal or pathologic myocardium. Also, given that thermal damage generally eliminates the observed birefringence resulting from myocardial muscle-fiber orientation, it is believed that PA imaging would be insensitive to fiber orientation.

In certain embodiments, the imaging is conducted in vitro.

In other embodiments, the imaging is conducted in vivo.

In certain embodiments, the imaging is a real time (RT) imaging.

In other embodiments, the imaging is a near-RT imaging.

In another embodiment, the method further comprising distinguishing an ablated portion from a non-ablated portion of the tissue.

The present invention can be used to determine prominent optical absorbers and characterize ablated and non-abated tissue during tissue ablation. Suitable absorbers include, but are not limited to deoxygenated hemoglobin, oxygenated hemoglobin, and spectra derived from ablated tissue.

Typically, during tissue ablation, physicians rely on indirect or bulk measurements of tissue properties to characterize ablated and non-ablated tissue (tissue-surface temperature, bulk tissue impedance, etc.). Photoacoustic imaging probes the optical properties of the tissue directly to image tissue optical absorption (molecular imaging) to identify which tissue has been ablated and which has not.

In another embodiment, the method can be used to visualize myocardial and other tissue ablation lesions.

In general, any tool used to guide ablation must be RT or near-RT. PA imaging frame rate is practically limited by the pulse repetition frequency of the irradiating laser source (10-20 Hz in this initial setup). Our initial system is capable of providing near-RT imaging (1-2 fps each consisting of 11 optical wavelengths with no averaging), and many laser systems, operating at kHz frequencies, are currently available that could provide RT PA imaging. Once studies have demonstrated which wavelengths are optimal for in vivo imaging, a system could be constructed using several diode-pumped lasers, operating at kHz pulse repetition rates, to provide frame rates well in excess of 30 fps.

In an in vivo environment, the ablation and imaging substrate will be highly oxygenated tissue. Given that the optical absorption of oxy-Hb can be nearly an order of magnitude less in the NIR regime, when compared to deoxy-Hb, one could expect that the background signal may be lower in highly oxygenated tissue. While this may require modification of the wavelengths chosen to image, if the spectrum of ablated tissue remains unchanged in vivo, then an in vivo environment could provide greater PA signal contrast between ablated and non-ablated tissue.

In vivo applications of PA imaging have already demonstrated feasibility of PA imaging through luminal blood using relatively low optical fluences and observing minimal signal from blood. An ICE implementation of PA imaging could achieve higher fluences than previous studies, and optical scattering due to blood may aid in homogenizing optical fluence at the endocardial surface. As with other clinical imaging modalities (US, MRI, CT), PA imaging can be implemented using a cardiac gating function to minimize motion between frames as well to reduce optical changes resulting from the cardiac perfusion cycle. Although the technical integration of an intracardiac probe capable of concurrent ablation and PA imaging will not be trivial, the development of such a clinical probe should be possible given recent advances in combined RFA-US catheters and the development of light delivery mechanisms utilized for intracardiac laser ablation.

In another embodiment, the present invention can be used to guide trans-catheter ablation of atrial arrhythmia.

In another embodiment, the temperature-induced changes of the photoacoustic signal from said tissue can be used to estimate tissue temperature.

By using the temperature dependence of the mechanism of photoacoustic signal generation, tissue temperature can be directly inferred, even at several millimeters of depth. assessment of tissue temperature during energy application can be used to monitor tissue temperature to reduce complications.

By supplying the physician with accurate molecular and temperature information during a procedure, the present invention can guide ablation procedures to both improve procedural efficacy and reduce procedural complications.

Additionally, due to the temperature dependence of β(T) (Equation 1), thermographic PA (tPA) mapping with high thermal and temporal resolutions (<1 K and ˜1 s, respectively) is possible. Guidance of ablation procedures represents an ideal application of tPA mapping as PA imaging has the potential to provide thermographic information co-registered with anatomical (US) and molecular (sPA) information. This feature of PA imaging may be used for laser, RF- and cryo-ablation guidance.

In another embodiment, during pacemaker/ICD lead placement, the multi-wavelength PA imaging can characterize the myocardial substrate to determine if it is a suitable position for lead placement.

In certain embodiments, the present invention can identify scar tissue from previous procedures or from damaged tissue.

The present invention can be used to directly characterize the tissue adjacent to pacing leads, rather than rely on electrogram studies to assess tissue conductivity. The present invention can also be used to identify sites where pacemaker lead conduction would likely be affected by scar tissue.

To facilitate a better understanding of the present disclosure, the following examples of certain aspects of some embodiments are given. In no way should the following examples be read to limit, or define, the entire scope of the invention.

### EXAMPLES

### Sample Preparation and Ablation

Fresh porcine hearts (Sierra for Medical Science, Whittier, Calif.) were acquired within 24 hours of sacrifice and were never frozen. The ventricles were harvested and samples were excised from these portions to produce approximately 20×20×10 mm³ sized specimens for ablation. The ablation system consisted of a Stockert 70 RF generator combined with a COOLFLOW® irrigation pump and a THERMOCOOL® irrigated tip catheter (Biosense Webster Inc., Diamond Bar, Calif.). During each ablation, the catheter was flushed with PBS at a rate of 10 ml min⁻¹. RF energy was applied at a rate of 20-30 W for 40-60 s. Tissue samples were submerged in normal PBS during ablation and imaging. After the ablation, samples were patted dry and returned to an airtight container (to minimize desiccation) and refrigerated.

### Imaging System Setup and Procedure

Normal PBS was used to acoustically couple the imaging system with the tissue. Imaging was performed on a combined PA/US imaging system that consisted of a Vevo® 2100 US imaging system (FUJIFILM VisualSonics Inc., Toronto, ON, Canada) paired with an LZ-250 transducer (21-MHz center frequency) with integrated fiber optics connected to a pulsed, tunable Nd:YAG laser (680-970 nm wavelength range). A single (i.e. no averaging) three-dimensional (3-D) combined PA/US B-mode scan was performed on each sample at 710 nm, which was the wavelength for peak laser energy (22 mJ per pulse). From that 3-D volume, a single 2-D plane corresponding to the brightest region of the PA signal was selected for sPA imaging from 680 -840 nm (20-14 mJ per pulse) in 2-nm steps. Ten PA frames at each wavelength were acquired and averaged into a single PA/US image at each sampled wavelength (FIGS. 1, 2A and 3B). Energy was measured at the fiber bundle output using an external power meter. This protocol was used to image eight ablated samples harvested from two porcine hearts. Later, a full 3-D PA/US volume was acquired for each wavelength from 740-780 nm (12-15 mJ/cm²) in 5-nm steps. At each wavelength, six matched PA/US volumes were acquired and averaged to reduce noise. The resulting 3-D sPA data were normalized to the average laser energy at each corresponding wavelength. This second protocol was used to image six ablated samples from two additional porcine hearts. A schematic of the imaging system is shown in FIG. 1.

### Sample Staining Procedure

For the purpose of sample staining, nitro-tetrazolium blue (NTB) salt (Sigma-Aldrich Corp., St. Louis, Mo.) was chosen to identify macroscopic myocardial tissue necrosis. The NTB solution was prepared by dissolving NTB in normal PBS at 0.5 mg ml⁻¹, as outlined by Ramkissoon. All specimens were incubated for 15 minutes in NTB solution maintained at 35° C. Specimens were then patted dry and photographed for gross pathology.

### ROI Selection and Analysis

Equal-sized region of interests (ROIs; 0.04 mm²) of the PA signal center (Ablated) and a specimen region external to the lesion (Non-ablated) were selected from the 2-D sPA dataset for analysis, as shown in the white boxes in FIG. 2A. The ROIs were chosen to be at both equivalent depths within the tissue and equivalent distances from the transducer to maintain similar optical fluences at each ROI.

### Contrast and Reference Spectra

$$C B R = \frac{{\overline{I}}_{Abl} - {\overline{I}}_{Nabl}}{{\overline{I}}_{Nabl}} \qquad Eq . 2$$

Contrast-to-background ratio (CBR) was calculated using Equation 2, where Ī_{Abl} and Ī_{Nabl} represent the mean PA signal intensity for the Ablated and Non-ablated ROI, respectively. This was done at 710 nm, the wavelength of peak laser energy. To obtain PA signal spectra, the mean PA intensities for both ROIs were calculated and plotted as a function of wavelength; FIG. 2B shows representative spectra observed in Ablated (red) and Non-ablated (blue) myocardium (normalized for display purposes). These spectra differ considerably near 760 nm, where the Non-ablated spectrum has a prominent hump, which correlates well with the extinction spectrum of Hb and is absent from the Ablated spectrum. For this reason, two spectra (FIG. 2D) were selected for a Pearson correlation test to characterize the two tissue types. Ablated reference spectra was obtained by averaging ROIs from ablated samples (n=8) harvested from two separate hearts. The known extinction spectrum for Hb was normalized and used as a reference for the non-ablated tissue.

### sPA Image Processing and Correlation

Prior to the correlation test, each sPA dataset was filtered spatially and spectrally (740-780 nm range) to reduce noise. Each 2-D sPA data frame was first filtered using a 0.22×0.19 mm² (Lateral×Axial) sliding average kernel at a specific wavelength. Then each pixel spectrum (740-780 nm) was filtered using a 6-nm wide median filter. For the 3-D sPA data, each 3-D volume was filtered using a 0.40×0.33×1.12 mm³ (Lateral×Axial×Elevation) sliding average kernel. No spectral filtering was applied to the 3-D sPA data. Voxel-by-voxel correlation, using both the Ablation-reference and the Hb-reference spectra, was performed on each 2-D and 3-D sPA dataset through 740-780 nm. For each sPA dataset, Ablation reference and Hb reference correlation maps were obtained. A final tissue characterization map (TCM, FIG. 2C) was then generated. Voxels of the TCM showing high (r>0.65) Ablation-reference or Hb-reference correlation were displayed as either red or blue, respectively, whereas voxels showing high correlation to both spectra were color coded green. TCM voxels showing poor correlation (r<0.65) to both spectra were not color coded. For each voxel that correlated to either Ablation or Hb reference spectra, the p-value of the correlation was calculated based on the null hypothesis. Three-dimensional rendering of the data (FIGS. 4A and 4B) was achieved with Amira (VSG, Burlington, Mass.); a clipping plane was introduced at the location of the tissue bisection (FIG. 4B) so that the 3-D rendered data could be compared with the side-view gross pathology (FIG. 4D).

### Image Registration and Comparison

Three-dimensional TCM/US data was co-registered with matching photographed gross pathology. Gross pathology photographs were acquired in top-view and side-view orientations. Both orientations were cropped and centered so that the gross pathology field of view (FOV) represented the same FOV as the US volume. In the top-view gross pathology images, tissue sample boundaries were manually segmented from the photograph background (FIG. 3A, blue). The ablated-region was manually segmented from the non-ablated tissue in the gross pathology images (FIG. 3A, red). Finally, a line was placed to identify the tissue bisection plane. These segmentations and line placement were repeated three times, independently, to reduce variability. An equivalent top-view orientation was then reconstructed using the TCM/US volume data. Matching segmentations were performed on this reconstructed view (also repeated three times, independently, on each sample).

A straightforward rigid image registration was applied to both the top-view and the side-view orientation image sets; each gross pathology image was resampled to match the TCM/US scan line density, and the TCM/US data was then registered with the gross pathology by translating the boundary segmentation centroids (FIG. 3A, blue) to be overlaid. The TCM/US segmentation was then rotated to find the maximum area overlap. Once the top-view gross pathology and matched TCM/US data were co-registered, an image slice was selected from the TCM/US data that corresponded to the tissue bisection plane. A similar segmentation and co-registration process was performed on the resulting side-view gross pathology and TCM/US data (FIGS. 3C and 3D, respectively).

Once both the top-view and side-view orientations were co-registered, the accuracy of the TCM was determined. The centroids of the ablated-region segmentations were calculated for each gross pathology and TCM set of both the top- and side-view orientations. For each orientation and sample, the lateral, axial and elevation offsets between the centroids were measured. Agreement between the segmentations was assessed by comparing the maximum axial, lateral or elevation extent of the segmented region. The % Area Agreement was defined as the area of the segmentations' intersection divided by the area of the segmentations' union,

$$\% Area Agreement ≡ \frac{{S}_{GP} ⋂ {S}_{TCM}}{{S}_{GP} ⋃ {S}_{TCM}} , \qquad Eq . 3$$

where S_{GP} and S_{TCM} represent the segmented ablated region from the gross pathology and TCM, respectively. For the side-view co-registered samples, two samples were cut parallel to the elevation axis, and four samples were cut parallel to the lateral axis, such that two samples were utilized to assess elevation offset, while four samples were utilized to assess lateral offset; all six side-view co-registered samples were utilized to assess axial offset.

### Results

### Single-wavelength Analysis

FIG. 2A shows a representative single-wavelength combined PA/US image acquired at 710 nm. Based on matched gross pathology images of the stained sample, the red-orange region located at the image center, at approximately 11 mm depth, correlates to the core of the lesion. For FIG. 2A, Ī_{Abl}=−7.8±3.0 dB, Ī_{Nabl}=−38.6±2.4 dB (both normalized to the PA signal peak intensity at 710 nm), and CBR is 30.7±3.1 dB.

### Spectroscopic Analysis

Both normalized spectra from the ROIs in FIG. 2A are shown in FIG. 2B. The Ablated absorption spectrum monotonically decreases, which agrees with results obtained by Swartling, et al. The Non-ablated absorption spectrum agreed well with that of Hb, notably displaying a prominent hump near 760 nm. In this wavelength range, Hb is expected to be the dominant absorber in a harvested (i.e. oxygen depleted) myocardial sample.

An example of the TCM overlaying the corresponding B-mode US image is displayed in FIG. 2C. The correlation maps demonstrate the ability to visualize both ablated and non-ablated tissue at greater depth and extent than that afforded by single-wavelength PA images. FIG. 2C indicates, at lateral positions of 6 mm and 10.5 mm, the correlation protocol was able to identify 2-3 mm of ablated myocardium beneath at least an additional millimeter of non-ablated myocardium. This was typical of both 2-D and 3-D imaged samples, where the sPA data showed good correlation to absorbers up to a depth of 3 mm. The average correlation p-value over all voxels was 0.0104 ±0.0098, suggesting that the overall correlation was highly significant.

### Lesion Dimension Statistics

TABLE 1
Comparison of Segmentation Metrics from Top- and side-view Gross
Pathology and TCM/US Registrations.
Lesion dimension statistics (n = 6 samples total)

| | Top-view orientation | | | Side-view orientation | | | |
|---|---|---|---|---|---|---|---|
| | Lateral | Elevation | Segmented | Axial | Lateral | Elevation | Segmented |
| | (mm) | (mm) | Area (mm²) | (mm) | (mm) | (mm) | Area (mm²) |
| TCM offset | 0.4 ± 0.4 | 0.0 ± 0.7 | — | 1.2 ± 0.8 | 0.1 ± 0.7 | 0.9 ± 1.5 | — |
| TCM extent | 7.4 ± 1.8 | 7.3 ± 1.6 | 37.8 ± 15.8 | 3.4 ± 1.0 | 8.0 ± 1.4 | 6.3 ± 1.9 | 14.5 ± 4.9 |
| Gross pathology | 6.5 ± 1.9 | 8.3 ± 1.5 | 41.8 ± 16.4 | 5.3 ± 1.0 | 7.8 ± 1.4 | 10.9 ± 3.7 | 30.7 ± 10.1 |
| extent | | | | | | | |
| Area agreement | — | — | 69 ± 11% | — | — | — | 36 ± 18% |

The results of the 3-D segmentation comparison are shown in Table 1 above. For the top-view orientation (FIGS. 3A and 3B), good agreement between the gross pathology images and TCM was achieved, with no elevation and low lateral (0.4 mm) offset measured. The absolute difference between the average lateral and elevation extent was 1 mm or less, and well within the standard deviations for both measurements. The mean segmented lesion areas were calculated to be 37.8±15.8 mm² and 41.8±16.4 mm² for the TCM and gross pathology, respectively. The difference between these areas was 4.0 mm² (approximately 10% total lesion area), which is within the standard deviations of both measurements. The % Area Agreement, as defined in Equation 3, was approximately 70% averaged across all 6 samples, indicating good TCM and gross pathology agreement for all 3-D volumes.

For the side-view orientation (FIGS. 3C and 3D), the two samples cut parallel to the elevation axis showed an average of a 0.9-mm offset in that dimension. The samples cut laterally showed good lateral agreement (offset of 0.1 mm). The axial dimension showed the greatest offset, averaging 1.2 mm across all 6 samples. The average axial extent of the lesion segmentations was 3.4 35 1.0 mm and 5.3±1.0 mm, for the TCM and gross pathology, respectively. Mean segmented areas were calculated to be 14.5±4.9 mm² and 30.7±10.1 mm² for the TCM and gross pathology, respectively, while the % Area Agreement was 36% (averaged across 6 samples).

The 3-D rendered data compared well with the matching gross pathology (FIGS. 4A-D). In this data set, there are two distinct regions of ablated tissue surrounded by non-ablated tissue and separated by an approximately 2-mm non-ablated gap (FIG. 4A). The tissue is also consistently identified to depths greater than 2 mm, for both ablated and non-ablated myocardium, and generally agrees well with the matched gross pathology (FIGS. 4C and 4D).

The features and advantages of the present disclosure will be readily apparent to those skilled in the art. While numerous changes may be made by those skilled in the art, such changes are within the spirit of the invention.

What is claimed is:

1. A method comprising:
providing a thermally ablated tissue; and
imaging the tissue with more than one wavelength of electromagnetic radiation capable of generating an acoustic signal.

2. The method of claim 1, further comprising identifying which tissue has been ablated.

3. The method of claim 1, further comprising thermally ablating a tissue.

4. The method of claim 1, wherein the wavelength is near-infrared.

5. The method of claim 1, wherein the imaging further comprises generating a tissue characterization map.

6. The method of claim 1, wherein the tissue is a liver tissue.

7. The method of claim 1, wherein the tissue is a prostate tissue.

8. The method of claim 1, wherein the tissue is a cardiac tissue.

9. The method of claim 1, further comprising imaging the tissue with a single-wavelength photoacoustic imaging.

10. The method of claim 1, wherein the imaging is conducted in vitro.

11. The method of claim 1, wherein the imaging is conducted in vivo.

12. The method of claim 1, wherein the imaging is a real time (RT) imaging.

13. The method of claim 1, wherein the imaging is a near-RT imaging.
